(19) United States
(12) Patent Application Publication
(10) Pub. No.: US 2004/0223895 A1
(43) Pub. Date: Nov. 11, 2004

(54) Perfume-dispensing adapter for use in a bubble generator for SPA

(76) Inventor: Shu Chih Wu, Chung Ho (TW)

Correspondence Address: PRO-TECHTOR INTERNATIONAL, 20775 Norada Court, Saratoga, CA, 95070-3018, US

(21) Appl. No.: 10/430,251

(22) Filed: May 5, 2003

(51) Int. Cl. A61L 9/00; A62B 7/08

(52) U.S. Cl. 422/305; 422/306; 422/123

(57) ABSTRACT

A perfume-dispensing adapter for use in a bubble generator for SPA is constructed to include a casing connected between a compressed air source and an air nozzle panel being put in a bathtub for guiding compressed air from the compressed air source into the air nozzle panel for producing air bubbles in the bathtub, a valve supported on a spring member in the casing and adapted to close the casing when the casing receiving no compressed air or to open the casing when the casing receiving compressed air, and a perfume dispenser mounted in the casing and adapted to emit a smell into compressed air passing through the casing.

### BACKGROUND OF THE INVENTION

1. Field of the Invention

The present invention relates to a SPA apparatus and, more specifically, to a perfume-dispensing adapter for use in a bubble generator for SPA.

2. Description of the Related Art

SPA is a mineral spring resorted to for cures or a locality containing a mineral spring resorted to for cures. The word of SPA comes from SolusPar Aqua. Nowadays, most fashionable resort localities and hotels provide mineral spring for cures. There are many commercially available home apparatus for producing mineral spring or bubbles for cures. Generating bubbles in water in a bathtub effectively massage the user's muscles. Further, while enjoying a SPA in a bathtub, people may pour fragrant oil in the bathtub. Because fragrant oil is not soluble in water, it floats in water in the bathtub, and the user's body will be covered with a layer of fragrant oil after SPA. Further, applied fragrant oil tends to be adhered to the surface of the bathtub, resulting in a bathtub cleaning problem.

### SUMMARY OF THE INVENTION

The present invention has been accomplished under the circumstances in view. It is one object of the present invention to provide a perfume-dispensing adapter, which is practical for use in a bubble generator for SPA. It is another object of the present invention to provide a perfume-dispensing adapter, which emits a good smell into compressed air passing through for producing bubbles in water when installed in a bubble generator. To achieve these and other objects of the present invention, the perfume-dispensing adapter comprises a casing, the casing having a rear end connected to a compressed air source, a front end connected to the air tube of an air nozzle panel put in a bathtub, and an air passage adapted to guide compressed air from the compressed air source into the air nozzle panel for producing air bubbles in the bathtub; a valve mounted in the casing and adapted to close/open the air passage subject to the presence of compressed air in the casing; a spring member mounted in the casing and supporting the valve in a close position closing the air passage; and a perfume dispenser mounted in the rear end of the casing, the perfume dispenser holding a flake perfume for emitting a smell into compressed air passing through the air passage of the casing.

### BRIEF DESCRIPTION OF THE DRAWINGS

FIG. 1 is a cutaway of the preferred embodiment of the present invention.

FIG. 2 is an exploded view of the preferred embodiment of the present invention.

FIG. 3 is an exploded view of the perfume dispenser according to the present invention.

FIG. 4 is sectional elevation of the preferred embodiment of the present invention, showing valve moved forwards, the spring member compressed.

FIG. 5 is a schematic drawing of the preferred embodiment of the present invention, showing the air path through the casing.

FIG. 6 is an applied view of the preferred embodiment of the present invention.

### DETAILED DESCRIPTION OF THE PREFERRED EMBODIMENT

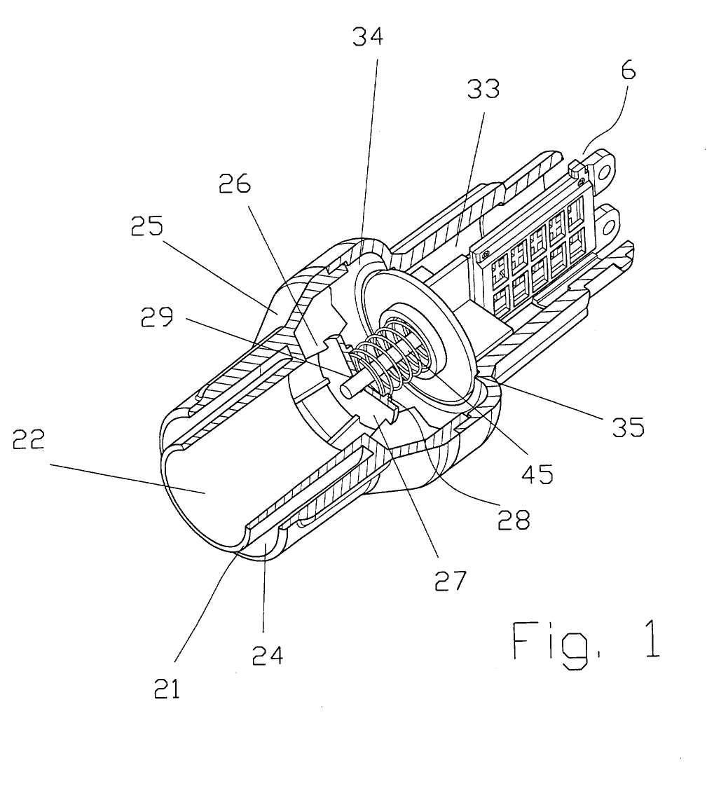

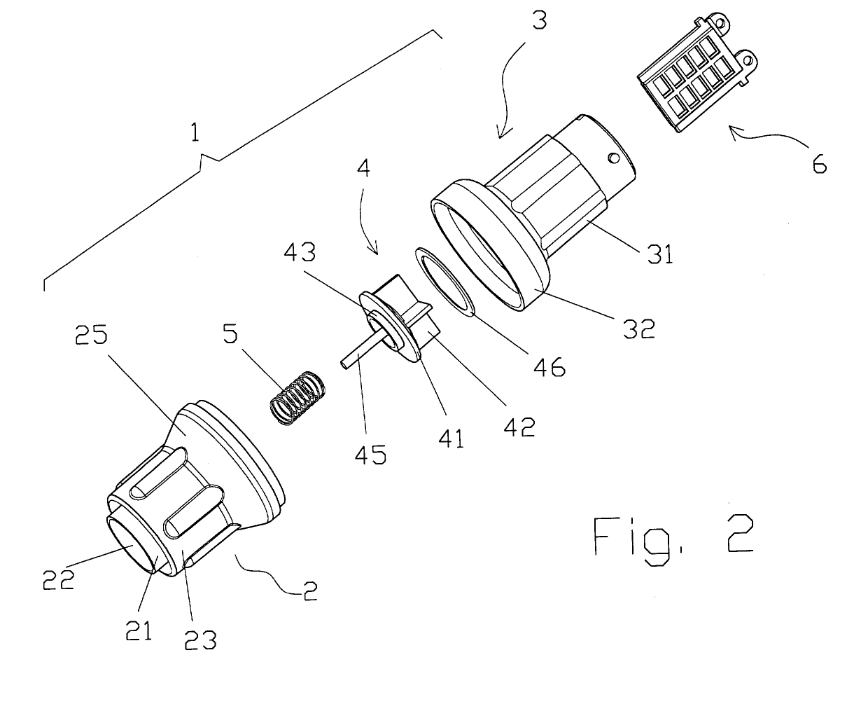

Referring to FIGS. 1 and 2, a perfume-dispensing adapter for use in a bubble generator for SPA in accordance with the present invention is shown comprised of a casing 1, a valve 4 mounted inside the casing 2, a spring member 5, which supports the valve 4 in the casing 1, and a perfume dispenser 6 mounted in one end, namely, the rear end of the casing 1.

The casing 1 is comprised of a front-half shell 2 and a rear-half shell 3. The front-half shell 2 comprises a coupling portion 25, an actuation chamber 26 defined within the coupling portion 25, an inner tube 21 extended from the coupling portion 25, a longitudinal air passage 22 defined within the inner tube 21, an outer tube 23 The rear-half shell 3, an outer tube 23 extended from the coupling portion 25 and coaxially disposed around the inner tube 21, a coupling groove 24 defined within the outer tube 23 around the inner tube 21, a partition wall 27 transversely formed in the coupling portion 25 between the air passage 22 and the actuation chamber 26, a recessed portion 28 formed in the center area of the partition wall 27, and a through hole 29 through the center of the recessed portion 28 in communication between the air passage 22 and the actuation chamber 26. The rear-half shell 3 comprises a tubular body 31, a coupling portion 32 extended from one end of the tubular body 31 and coupled to the coupling portion 25 of the front-half shell 2, a receiving chamber 33 defined in the tubular body 31, an actuation chamber 34 defined in the coupling portion 32, and a seat 35 defined in between the receiving chamber 33 and the actuation chamber 34.

The valve 4 is mounted in the actuation chambers 26 and 34 of the casing 1, comprising a valve flap 41 rested on the seat 35 inside the rear-half shell 3, a plurality of radial fins 42 located on one side, namely, the back side of the valve flap 41 and suspending in the receiving chamber 33 of the rear-half shell 3, an annular flange 43 located on the other side, namely, the front side of the valve flap 41, a recessed portion 44 defined within the annular flange 43 and adapted to accommodate the spring member 5, and a guide rod 45 perpendicularly forwardly extended from the center of the front side of the valve flap 41 and insertable into the through hole 29 of the front-half shell 2.

The spring member 5 is compression spring sleeved onto the guide rod 45 of the valve 4 and stopped between the recessed portion 28 of the front-half shell 2 and the recessed portion 44 of the valve 4.

Referring to FIG. 3 and FIGS. 1 and 2 again, the perfume dispenser 6 is mounted in the receiving chamber 33 of the rear-half shell 3, comprised of two frame shells 61 and 62 fastened together, and a flake perfume 65 held in between the frame shells 61 and 62. The frame shells 61 and 62 have open spaces 63 and 64 through which the good smell of the flake perfume is dissipated into the air passing through the casing 1.

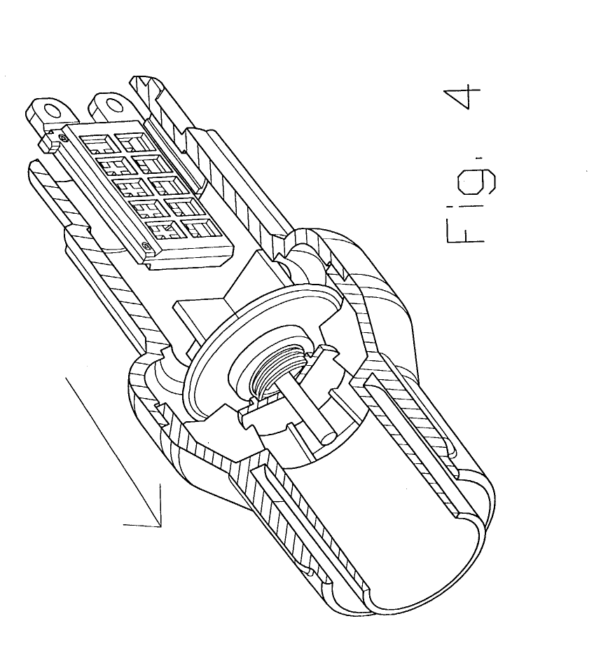

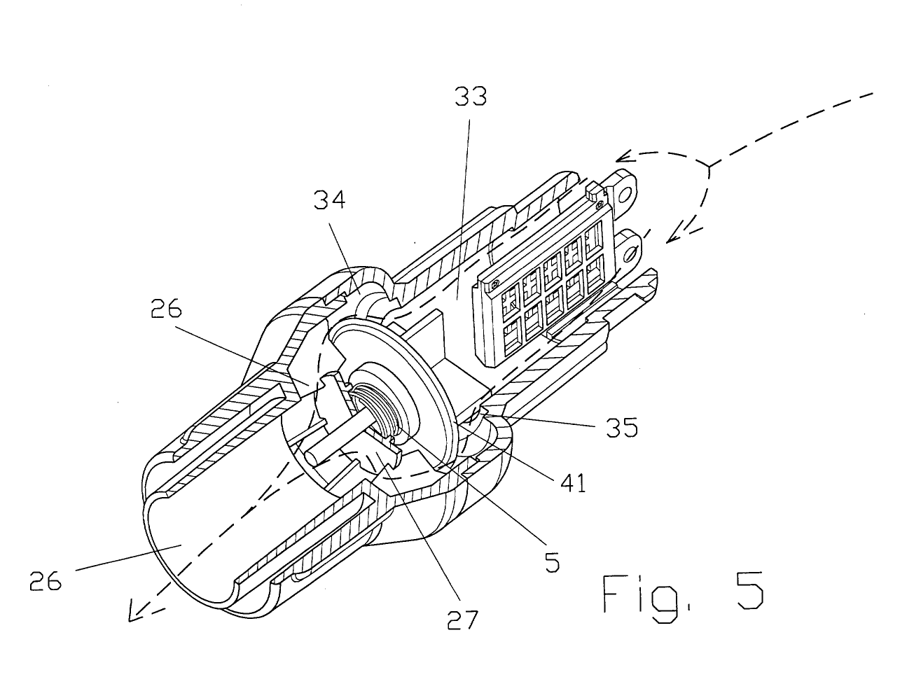

Referring to FIGS. 4 and 5, when a flow of air guided into the receiving chamber 33 of the rear-half shell 3, the intake flow of air pushes the valve 4 forwards against the spring member 5, thereby causing the valve flap 41 to be moved away from the seat 35 for enabling the intake flow of air to pass from the receiving chamber 33 into the actuation chambers 34 and 26 and then to the outside of the front-half shell 2 via the air passage 22. At the same time, the good smell of the flake perfume 65 is carried with the intake flow of air to the outside of the casing 1.

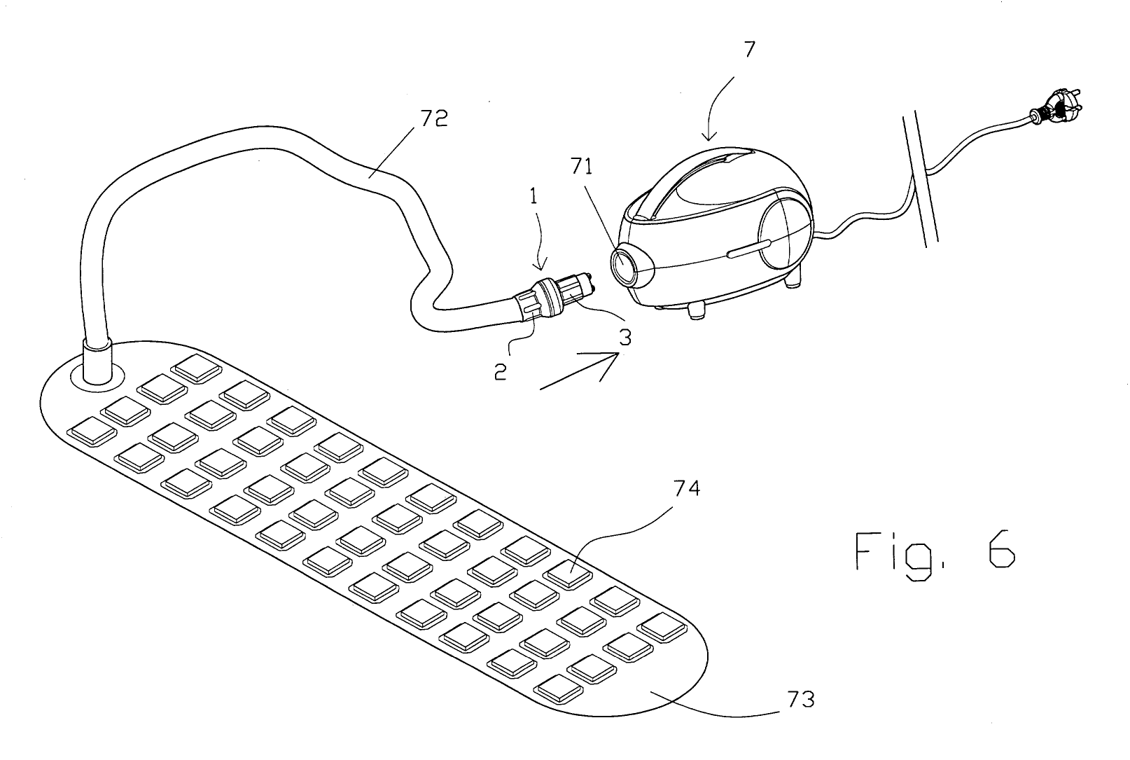

Referring to FIG. 6 and FIGS. 1 and 3 again, when in use, the air tube 72 of an air nozzle panel 73 having air nozzles 74 is connected to the coupling groove 24 of the casing 1, and then the air outlet 71 of a fan motor 7 is connected to the tubular body 31 of the rear-half shell 3 of the casing 1. When the fan motor 7 started, currents of air are induced and forced through the casing 1 to carry the good smell of the flake perfume 65 from the perfume dispenser 6 out of the casing 1 into the air nozzle panel 73 via the air tube 72. When currents of air passed out of the air nozzles 74 of the nozzle panel 73 into the water in the bathtub in which the air nozzle panel 73 is placed, air bubbles with good smell are produced.

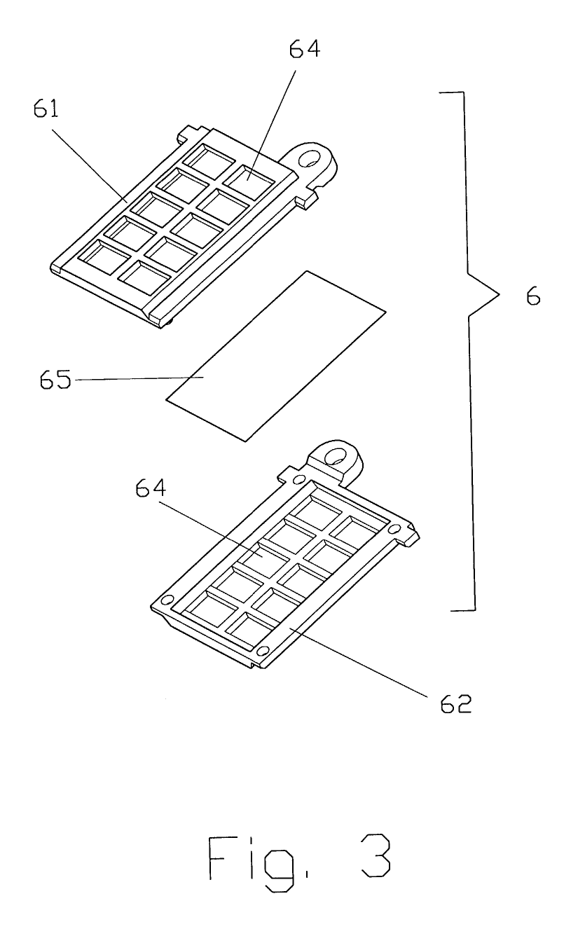

Referring to FIG. 3 again, the frame shells 61 and 62 of the perfume dispenser 6 are preferably injection-molded from plastics. Further, the frame shells 61 and 62 each have one end formed integral with each other when molded.

A prototype of perfume-dispensing adapter has been constructed with the features of FIGS. 1˜6. The perfume-dispensing adapter functions smoothly to provide all of the features discussed earlier.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A perfume-dispensing adapter for use in a bubble generator for SPA, comprising:
a casing, said casing having a rear end connected to a compressed air source, a front end connected to the air tube of an air nozzle panel put in a bathtub, and an air passage adapted to guide compressed air from the compressed air source into the air nozzle panel for producing air bubbles in the bathtub;
a valve mounted in said casing and adapted to close/open said air passage subject to the presence of compressed air in said casing;
spring means mounted in said casing and supporting said valve in a close position closing said air passage; and
a perfume dispenser mounted in the rear end of said casing, said perfume dispenser comprising two open frame shells and a flake perfume held in between said open frame shells for emitting a smell into compressed air passing through the air passage of said casing.

2. The perfume-dispensing adapter as claimed in claim 1, wherein said casing is comprised of a front-half shell and a rear-half shell.

3. The perfume-dispensing adapter as claimed in claim 2, wherein said front-half shell comprises a coupling portion for connection to said rear-half shell, an inner tube extended from the coupling portion of said front-half shell and defining said air passage, an outer tube extended from the coupling portion of said front-half shell, and a coupling groove defined within said outer tube around said inner tube and adapted to receive the air tube of the air nozzle panel.

4. The perfume-dispensing adapter as claimed in claim 3, wherein said front-half shell further comprises an actuation chamber defined within the coupling portion thereof, a partition wall transversely formed in the coupling portion of said front-half shell between said air passage and the actuation chamber of said front-half shell, a recessed portion formed in said partition wall, and a through hole through the center of said recessed portion in communication between said air passage and the actuation chamber of said front-half shell.

5. The perfume-dispensing adapter as claimed in claim 4, wherein said rear-half shell comprises a tubular body, a coupling portion extended from one end of said tubular body and coupled to the coupling portion of said front-half shell, a receiving chamber defined in said tubular body, an actuation chamber defined in the coupling portion of said rear-half shell, and a valve seat defined in between the receiving chamber and actuation chamber of said rear-half shell and adapted to accommodate said valve.

6. The perfume-dispensing adapter as claimed in claim 5, wherein said valve is mounted in the actuation chambers of said front-half shell and said rear-half shell, comprising a valve flap forced by said spring means to rest on said valve seat inside said rear-half shell, said valve flap having a front side and a rear side, a plurality of radial fins located the back side of said valve flap and suspending in the receiving chamber of said rear-half shell, an annular flange located on the front side of said valve flap, a recessed portion defined within said annular flange and adapted to accommodate one end of said spring means, and a guide rod perpendicularly forwardly extended from the center of the front side of said valve flap and inserted into the through hole of said front-half shell.

7. The perfume-dispensing adapter as claimed in claim 1, wherein said spring means is a compression spring.

8. The perfume-dispensing adapter as claimed in claim 1, wherein the open frame shells of said perfume dispenser each have one end formed integral with each other.
